(19) United States
(12) Patent Application Publication
(10) Pub. No.: US 2015/0329512 A1
(43) Pub. Date: Nov. 19, 2015

(54) METHOD AND PHARMACEUTICAL COMPOSITION FOR TREATING LUNG CANCER

(71) Applicant: National Dong Hua University, Huanlien (TW)

(72) Inventors: Ching-Feng Weng, Huanlien (TW); Yi-Chen Chia, Huanlien (TW); Wei-Jun Chiu, Huanlien (TW)

(73) Assignee: National Dong Hua University, Huanlien (TW)

(21) Appl. No.: 14/280,383

(22) Filed: May 16, 2014

(51) Int. Cl. C07D 307/33; A61K 31/341; A61K 45/06

(52) U.S. Cl. CPC C07D 307/33; A61K 45/06; A61K 31/341

(57) ABSTRACT

The present invention is related to a method and pharmaceutical composition for treating lung cancer. The pharmaceutical composition comprises an effective amount of 16-hydroxy-cleroda-3,13-dine-15,16-olide as active ingredient, and a pharmaceutically acceptable carrier. The present method and pharmaceutical composition provides good efficacy in treating lung cancer even for drug-resistance patient. The present invention also establishes an animal model bearing human lung cancer, which provides a better drug screening platform for the research.

### BACKGROUND

1. Technical Field

The present disclosure relates to a method and a pharmaceutical composition for treating lung cancer. In addition, the present invention relates to the establishment of animal model.

2. Description of Related Art

Lung adenocarcinoma is the top one cause of death due to cancer in Taiwan. Iressa, Taxol, Cisplatin, and Doxorubicin are conventionally used for treating lung adenocarcinoma. However, chemotherapy treatment is a factor causing lung adenocarcinoma metastasis and drug resistance frequently happens, which dramatically reduces the survival rate of lung cancer patients.

As appreciated the difficulty in treating lung adenocarcinoma and the conventional drugs more or less have the problem of resistance, it is the first priority goal of the clinic to find novel and effective drugs. Furthermore, there is always a distinct gap between laboratory experiments and clinical use; therefore, it is also an important task to imitate the laboratory experiments as similar as possible with a realistic pathophysiological condition in human beings. In this consideration, animal model is indeed taken as a significant step before the candidate drugs actually enter clinical trials. Of course, the animal model itself has still certain level of difference from human body. However, the appropriate animal model can provide a suitable glance to mimic for the exploration of human body disease.

In light of the foregoing, there is always a need for novel and useful drug for treating lung adenocarcinoma. In addition, it will be helpful for the drug screening if the conventional animal model can be modified to be more similar with human body.

### SUMMARY

One of the objects of the present invention is to provide a novel and useful drug for treating lung cancer by validating the medical use of 16-hydroxy-cleroda-3,13-dine-15,16-olide (HCD) in this regard.

Another object of the present invention is to provide a method for treating lung cancer, preferably the method can align the use of 16-hydroxy-cleroda-3,13-dine-15,16-olide with other conventional anti-cancer drugs to reduce the existing drug resistance and obtain better efficacy.

More an object of the present invention is to establish an animal model having pathophysiological condition much more similar with human.

In order to achieve the above-mentioned objects, the present invention provides a pharmaceutical composition for lung cancer treatment, comprising: an effective amount of 16-hydroxy-cleroda-3,13-dine-15,16-olide; and a pharmaceutically acceptable carrier.

Preferably, said effective amount is 0.01 to 1.28 mg/kg body weight.

Preferably, said composition comprises 2.1 to 25 !μM of said 16-hydroxy-cleroda-3,13-dine-15,16-olide.

Preferably, said lung cancer is squamous cell carcinoma, large-cell lung cancer, small-cell lung cancer and non-small-cell lung cancer, or a combination thereof.

Preferably, said lung cancer is resistant to Doxorubicin.

Preferably, said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

Preferably, an administration route of said composition is via oral administration, intravenous injection, intrathecal injection, or a combination thereof.

The present invention also provides a method for treating a lung cancer, comprising: administrating an object in need an effective amount of 16-hydroxy-cleroda-3,13-dine-15,16-olide.

Preferably, said effective amount is 0.01 to 1.28 mg/kg body weight.

Preferably, said administrating is via oral administration, intravenous injection, intrathecal injection, or a combination thereof.

Preferably, said 16-hydroxy-cleroda-3,13-dine-15,16-olide is administered with a pharmaceutically acceptable carrier

Preferably, said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

Preferably, said method further comprises a step after administrating said 16-hydroxy-cleroda-3,13-dine-15,16-olide: administrating said object with Doxorubicin.

Preferably, said lung cancer is an adenocarcinoma, a squamous cell carcinoma, a large-cell lung cancer, a small-cell lung cancer, a non-small-cell lung cancer, or a combination thereof.

The present invention more provides a method for establishing an animal model bearing human lung cancer: providing an animal; providing a cell suspension; wherein said cell is human lung cancer cell; and administrating said cell suspension into said animal's lung.

Preferably, said animal is not immunodeficient.

Preferably, said human lung cancer cell is a commercially-available cell line.

Preferably, said human lung cancer cell is resistant to an anti-cancer drug.

Preferably, said anti-cancer drug is Doxorubicin.

Preferably, said administering is via intrathecal injection.

Preferably, said administering is conducted with a dosage of 1×10⁵ to 1×10⁷ said cells per animal.

Preferably, said animal is rabbit, pig or rodent.

Preferably, said administering is repeated at least once.

To sum up, the present invention validates the medical use of HCD is in treating lung cancer and its ability to reduce the drug resistance of conventional anti-cancer drug. Moreover, the present invention also establishes an animal model that bearing human lung cancer so that the drug screening using said animal model can be more effective and reliable in subsequent clinical trials.

### BRIEF DESCRIPTION OF THE DRAWINGS

FIG. 1 shows the results of MTT assay. The MTT assay was performed for examining the effects of Dox on the cell viability of Anti-Dox-A549 cells and A549 cells at 24 hr treatment. All the experiments were done in three independent tests and the data was presented as mean±SD in triplicate determinations. Compared at *p<0.05, **p<0.01, ***p<0.001 levels with untreated control.

FIG. 2 shows the results of MTT assay. The MTT assay was performed for examining the effects of HCD on the cell viability of Anti-Dox-A549 cells and A549 cells at 24 or 48 hr treatment. All the experiments were done in three independent tests and the data was presented as mean±SD in triplicate determinations. Compared at *p<0.05, **p<0.01, ***p<0.001 levels with untreated control.

FIG. 3 shows the results of MTT assay. The MTT assay was performed for examining the effects of PG on the cell viability of Anti-Dox-A549 cells and A549 cells at 24 or 48 hr treatment. All the experiments were done in three independent tests and the data was presented as mean±SD in triplicate determinations. Compared at *p<0.05, **p<0.01, ***p<0.001 levels with untreated control.

FIG. 4 shows the results of MTT assay. The MTT assay was performed for examining the effects of combining pre-treatment of HCD is and follow-up treatment of Dox (1 μM) on cell viability of Anti-Dox-A549 cells and A549 cells. All the experiments were done in three independent tests and the data was presented as mean±SD in triplicate determinations. Compared at *p<0.05, **p<0.01, ***p<0.001 levels with untreated control.

FIG. 5 shows the results of MTT assay. The MTT assay was performed for examining the effects of combining pre-treatment of HCD and follow-up treatment of Dox (2.5 μM) on cell viability of Anti-Dox-A549 cells and A549 cells. All the experiments were done in three independent tests and the data was presented as mean±SD in triplicate determinations. Compared at *p<0.05, **p<0.01, ***p<0.001 levels with untreated control.

FIG. 6 shows the results of MTT assay. The MTT assay was performed for examining the effects of combining pre-treatment of PG and follow-up treatment of Dox (1 μM) on cell viability of Anti-Dox-A549 cells and A549 cells. All the experiments were done in three independent tests and the data was presented as mean±SD in triplicate determinations. Compared at *p<0.05, **p<0.01, ***p<0.001 levels with untreated control.

FIG. 7 shows the results of MTT assay. The MTT assay was performed for examining the effects of combining pre-treatment of PG and follow-up treatment of Dox (2.5 μM) on cell viability of Anti-Dox-A549 cells and A549 cells. All the experiments were done in three independent tests and the data was presented as mean±SD in triplicate determinations. to Compared at *p<0.05, **p<0.01, ***p<0.001 levels with untreated control.

FIG. 8 shows the lung morphology of the mouse model experiments in the Example 4 of the specification. (a) Normal mouse; (b) Mouse injected with 10% EtOH; (c) Mouse injected with Anti-Dox-A549 is cells; and (d) Mouse injected with A549 cells.

FIG. 9 shows the H&E and IHC staining conducted in the Example 4 of the specification. (a) Normal mouse; (b) Mouse injected with 10% EtOH; (c) Mouse injected with Anti-Dox-A549 cells; and (d) Mouse injected with A549 cells. “1” indicates blood vessels; “2” indicates cell accumulation; “3” indicates alveolus; “4” indicates trachea.

FIG. 10 shows the body weight changing curves of normal mouse, mouse injected with 10% EtOH, mouse injected with Anti-Dox-A549 cells, and mouse injected with A549 cells.

FIG. 11 shows the body weight changing curves of mouse bearing lung cancer after HCD or PG (as a positive) treatment.

FIG. 12 shows the H&E and IHC staining of the lung of mouse in Example 5 treated with HCD (0.0318 mg/kg body weight). (a) Mouse bearing lung cancer established by injection of Anti-Dox-A549 cells. (b) Mouse bearing lung cancer established by injection of A549 cells. “1” indicates blood vessels; “2” indicates cell accumulation; “4” indicates trachea.

FIG. 13 shows the H&E and IHC staining of the lung of mouse in Example 5 treated with HCD (1.272 mg/kg body weight). (a) Mouse bearing lung cancer established by injection of Anti-Dox-A549 cells. (b) Mouse bearing lung cancer established by injection of A549 cells. “1” indicates blood vessels; “2” indicates cell accumulation; “4” indicates trachea.

FIG. 14 shows the H&E and IHC staining of the lung of mouse in Example 5 treated with PG (0.0646 mg/kg body weight as a positive). (a) Mouse bearing lung cancer established by injection of Anti-Dox-A549 cells. (b) Mouse bearing lung cancer established by injection of A549 cells. “1” indicates blood vessels; “2” indicates cell accumulation; “4” indicates trachea.

is FIG. 15 shows the H&E and IHC staining of the lung of mouse in Example 5 treated with PG (0.1615 mg/kg body weight as a positive). (a) Mouse bearing lung cancer established by injection of Anti-Dox-A549 cells. (b) Mouse bearing lung cancer established by injection of A549 cells. “1” indicates blood vessels; “2” indicates cell accumulation; “4” indicates trachea.

### DETAILED DESCRIPTION

16-hydroxy-cleroda-3,13-dine-15,16-olide (HCD) isolated from Polyalthia longifolia possess some medicinal values; however, there is no evidence showing its value in treating lung cancer before the present invention.

The term “lung cancer” herein is referred to as an adenocarcinoma, such as, squamous cell carcinoma, large-cell lung cancer, small-cell lung cancer, non-small-cell lung cancer, or a combination thereof. It is important to note that the present invention found that HCD has efficacy in treating both of non-resistant lung cancer and drug-resistant lung cancer.

The term “treatment or treating” herein is referred to control or reduce the size of the tumor, prevent or limit the metastasis of the cancer cells, or a combination thereof. The term “effective amount” herein is referred to as an amount of the active ingredient that is sufficient to perform the aforesaid efficacies of treatment.

Said effective amount can be obtained from clinical trial (in human), animal model, or in vitro cell culture data. It is known in the field that the effective amount obtained from animal model or in vitro cell culture data can be calculated into the effective amount suitable for human use. For instance, as reported by Reagan-Shaw et al., 2008, “μg/ml” (effective amount based on in vitro cell culture experiments)=“mg/kg body is weight/day” (effective amount for mouse). Furthermore, the effective amount for mouse can be further modified based on the fact that the metabolism rate of mice is 6 times fast compared to human.

Said pharmaceutically acceptable carrier in the present invention includes but not limited to water, phosphate buffered saline, alcohol, glycerol, chitosan, alginate, chondroitin, Vitamin E, mineral oil, dimethyl sulfoxide (DMSO), or a combination thereof. Generally, the pharmaceutically acceptable carrier can be chosen based on the desired administration route, components of the drug, treatment strategies, or purposes to be met.

The first aspect of the present invention is to provide a pharmaceutical composition for lung cancer treatment. Said pharmaceutical composition comprises 16-hydroxy-cleroda-3,13-dine-15,16-olide (HCD) as the active ingredient. The effective amount of said HCD is 0.01 to 1.27 mg/kg body weight. The pharmaceutical composition can be administrated via oral administration, intravenous injection, intrathecal injection, or a combination thereof.

The second aspect of the present invention is to provide a method for treating a lung cancer, comprising: administrating an object in need an effective amount of 16-hydroxy-cleroda-3,13-dine-15,16-olide. The effective amount of said HCD is 0.01 to 1.27 mg/kg body weight. The administrating can be via oral administration, intravenous injection, intrathecal injection, or a combination thereof.

In a preferable embodiment of the present invention, a treating strategy is provided. Said treating strategy is to reduce the drug resistant level of a cancer cell concerned. Said treating strategy comprises a pre-treatment and a subsequent treatment. Said pre-treatment is administrating an object in need an effective amount of 16-hydroxy-cleroda-3,13-dine-15,16-olide. Said subsequent treatment is administrating said object with an anti-cancer drug that is known the cancer of said object has resistance to. After said pre-treatment, the data of the present invention showed that the efficacy of said anti-cancer drug can be rescued, which means the drug-resistance thereto was reduced.

Taking Doxorubicin (which is a known drug for chemotherapy, and it is known that some cancer patients have developed resistance to it during treatment) as an example, the present invention showed (in the following data) pre-treating with HCD can enhance the efficacy of the subsequent Doxorubicin treatment in drug-resistant cells. The data further showed that the pre-treatment of Prodigiosin (PG, as a postive) can also provide similar effects in reducing the drug-resistance of anti-Doxorubicin cells. Accordingly, the present invention indicates the potential of a co-treating strategy having a pre-treatment of HCD or PG and a subsequent treatment of a anti-cancer drug, even if the object has previously shown resistance to the anti-cancer drug in said subsequent treatment.

The third aspect of the present invention is to establish an animal model bearing human lung cancer. In this way, the drug screening data from said animal model can be more likely to be the things happened in human body because the animal has human lung cancer instead of mouse lung cancer.

The animal model of the present invention is established by administrating a cell suspension into an animal's lung. Said cell can be non-drug resistant cell or drug-resistant cell. If the animal model is established by drug-resistant cell, then the cancer borne in the animal could be also drug-resistant. In an alternative embodiment, said cell can be a commercially-available cell line.

In a preferable embodiment, the administrating can be made by intrathecal injection. In addition, said administrating is preferably repeated at least once; more preferably, repeated three times. Moreover, the dosage is of said administrating can be 1×10⁵ to 1×10⁷ cells per animal. In an alternative embodiment, said animal can be rabbit, pig or rodent. It is preferable that said animal is not immunodeficient, which means said animal is normal and immunological competence.

### EXAMPLE 1

### Experimental Design

### Reagent

The 16-hydroxy-cleroda-3,13-dine-15,16-olide (HCD) used in the present study was obtained from Professor Yi-Chen Chia (Department of Food Science & Technology, Tajen University, Taiwan). The Prodigiosin (PG) isolated from Serratia marcescens was obtained from Professor Jui-Hsin Su (Institute of Marine Biotechnology, National Drug Hwa University, Taiwan). The known drug for chemotherapy, Doxorubicin (Dox) was purchased from Sigma. Said HCD, PG, and Dox of various concentrations were dissolved in phosphate buffered saline (PBS) as indicated in the following paragraphs and were sterilized before use.

### Cells

A549 (lung cancer cell line, non-resistance) and Anti-Dox-A549 (lung cancer cell line with resistance to Doxorubicin) were maintained in DMEM supplemented with 10% fetal bovine serum (FBS, Gibco), pH 7.4, 37° C. and with continuous circulation of 5% CO₂. The medium was changed every 2 days and the cells were trypsinized with trypsin/EDTA when reaching 80%-90% confluence.

### Treatments

A549 and Anti-Dox-A549 were treated by the following treatment (Table 1) for 24 or 48 hr for examining the effects of HCD, PG, and Dox on their viability.

Dox1is referred as Dox of 1 μM; Dox2.5 is referred as Dox of 2.5 μM; Dox5 is referred as Dox of 5 μM; Dox10 is referred as Dox of 10 μM; Dox100 is referred as Dox of 100 μM. HCD2.5 is referred as HCD of 2.5 μM; HCD10 is referred as HCD of 10 μM; HCD25 is referred as HCD of 25 μM; HCD50 is referred as HCD of 50 μM. PG2.5 is referred as PG of 2.5 μM; PG5 is referred as PG of 5 μM; PG10 is referred as PG of 10 μM; PG20 is referred as PG of 20 μM.

TABLE 1
Treatment listing for the studies of the present invention

| Treatment | | | |
|---|---|---|---|
| No. | Pre-treated | Treated | Labeled |
| 1 | None | Dox2.5 | Dox2.5 |
| 2 | None | Dox5 | Dox5 |
| 3 | None | Dox10 | Dox10 |
| 4 | None | Dox100 | Dox100 |
| 5 | None | HCD2.5 | HCD2.5 |
| 6 | None | HCD10 | HCD10 |
| 7 | None | HCD25 | HCD25 |
| 8 | None | HCD50 | HCD50 |
| 9 | None | PG2.5 | PG2.5 |
| 10 | None | PG5 | PG5 |
| 11 | None | PG10 | PG10 |
| 12 | None | PG20 | PG20 |
| 13 | HCD2.5 | Dox1 | HxD1 |
| 14 | HCD2.5 | Dox2.5 | HxD2.5 |
| 15 | HCD10 | Dox1 | HyD1 |
| 16 | HCD10 | Dox2.5 | HyD2.5 |
| 17 | HCD25 | Dox1 | HzD1 |
| 18 | HCD25 | Dox2.5 | HzD2.5 |
| 19 | PG2.5 | Dox1 | PxD1 |
| 20 | PG2.5 | Dox2.5 | PxD2.5 |
| 21 | PG5 | Dox1 | PyD1 |
| 22 | PG5 | Dox2.5 | PyD2.5 |
| 23 | PG10 | Dox1 | PzD1 |
| 24 | PG10 | Dox2.5 | PzD2.5 |

### EXAMPLE 2

### Tests on Cell Viability

In this example, the effects of HCD, PG, and Dox on the viability of A549 and anti-Dox-A549 were examined. The MTT assay was employed for this purpose. The MTT (3-(4-,5-dimethylthiazol-2-yl)-2,5-diphenyltetrazolium bromide) assay is a common colorimetric method in the field for cell viability analysis. MTT (yellow tetrazolium salt) is reduced to a purple formazan by living cells and detection to the purple formazan can be calculated as the cell viability.

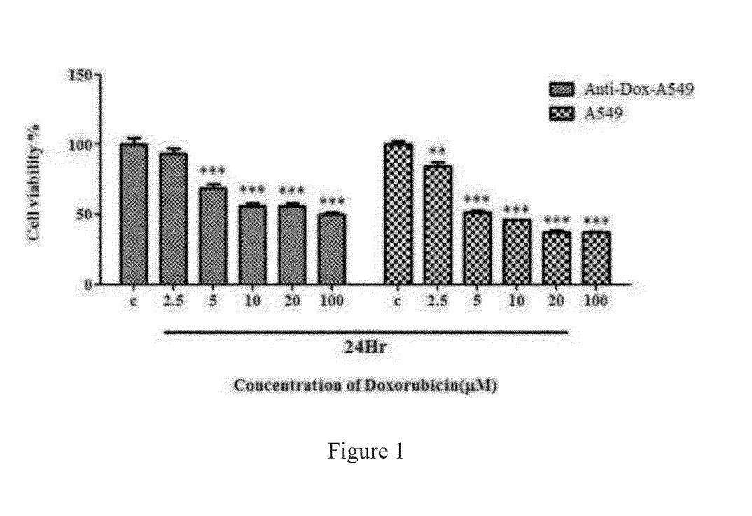

Briefly, cells (A549 or Anti-Dox-A549) were seeded in 96-well plate (7×10³ cell per well) and incubated overnight (37° C., 5% CO₂). Then, cells were treated in accordance with the treatments listed in the Table 1. The results were showed in FIGS. 1, 2, 3, 4, and 5.

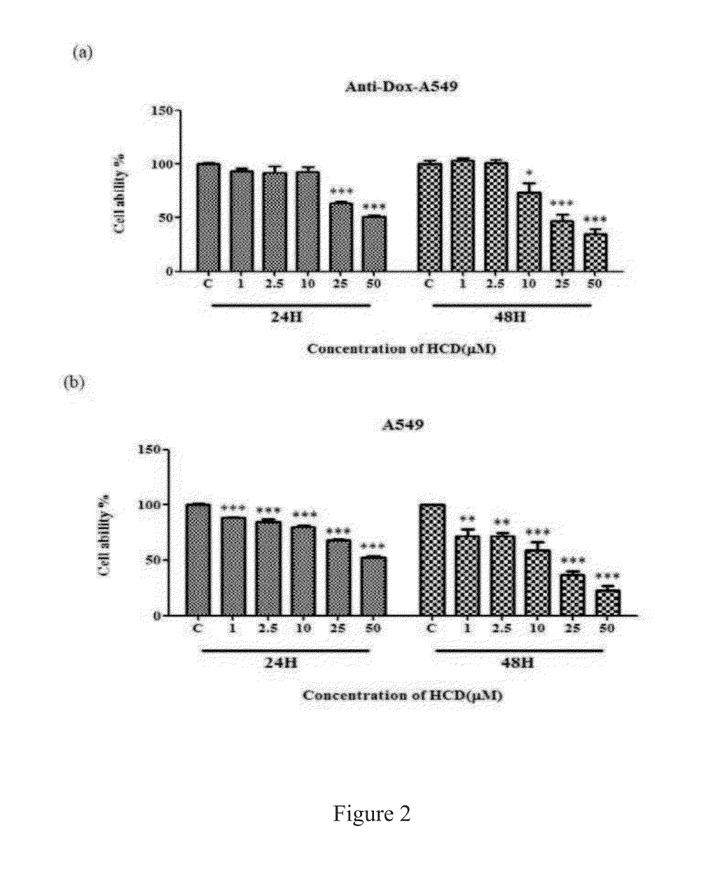

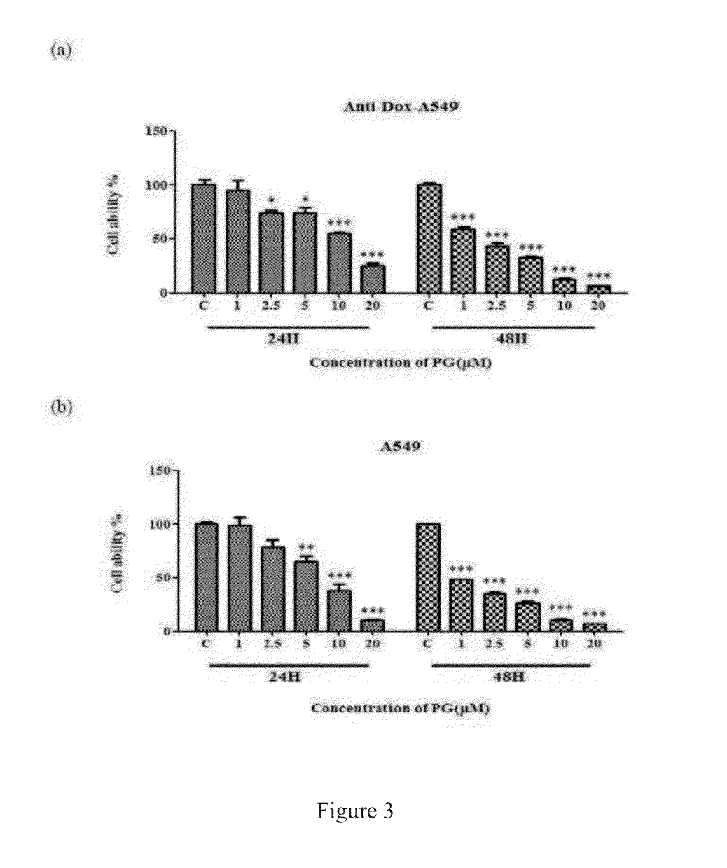

FIG. 1 showed the cell viability after Dox treatment. The IC₅₀ of Dox of Anti-Dox-A549 cells was about 100 μM; whereas, the IC₅₀ of A549 cells was 5 to 10 μM showing that the Dox-resistance in this cell line. The IC₅₀ showed no significant difference between A549 cells and Anti-Dox-A549 cells in HCD; wherein the IC₅₀ of A549 and Anti-Dox-A549 in HCD treatment are about 50 μM (FIG. 2, 24 hr). In terms of PG treatment, the IC₅₀ of A549 cells was slightly lower than that of Anti-Dox-A549 cells, indicating that Anti-Dox-A549 cells also exhibited some level of resistance to PG (FIG. 3). The data in FIG. 2 and FIG. 3 also showed that the treating period (24 or 48 hr), was also a factor to the cell viability. Basically, the cell viability of every treatment exhibited a dose-dependent manner.

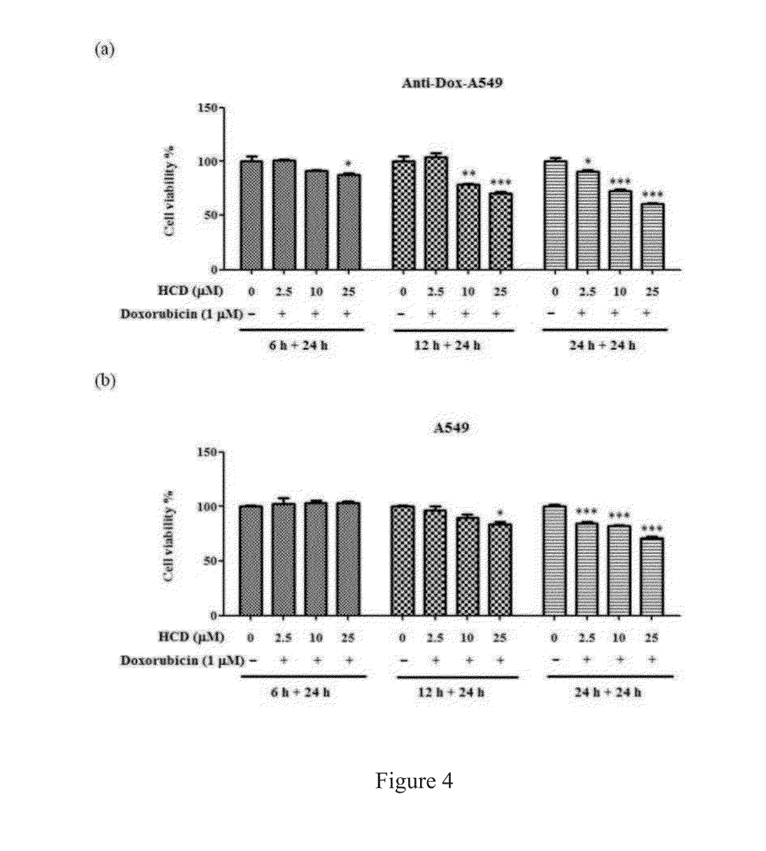

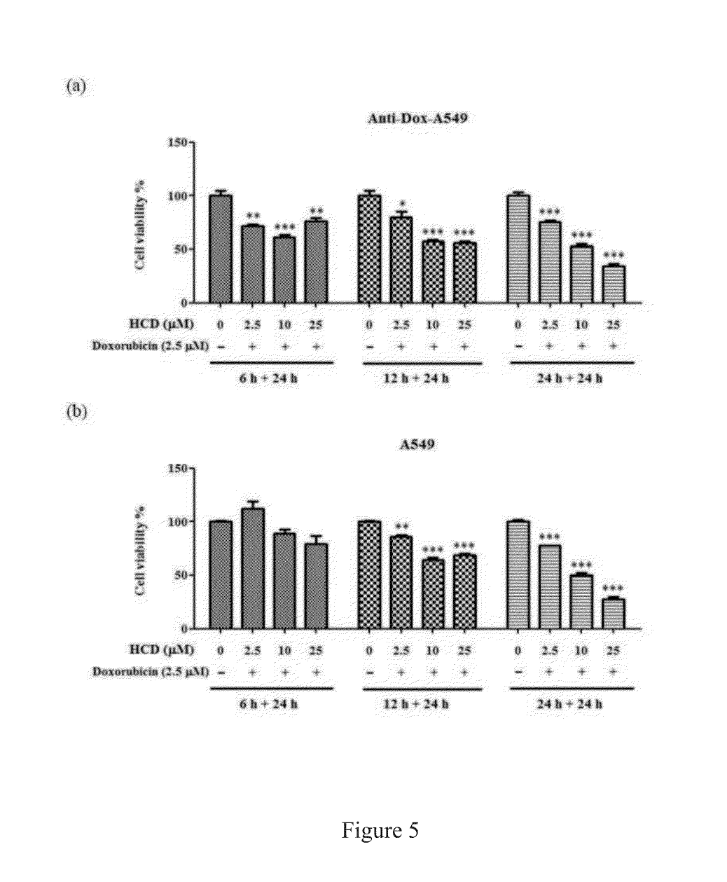

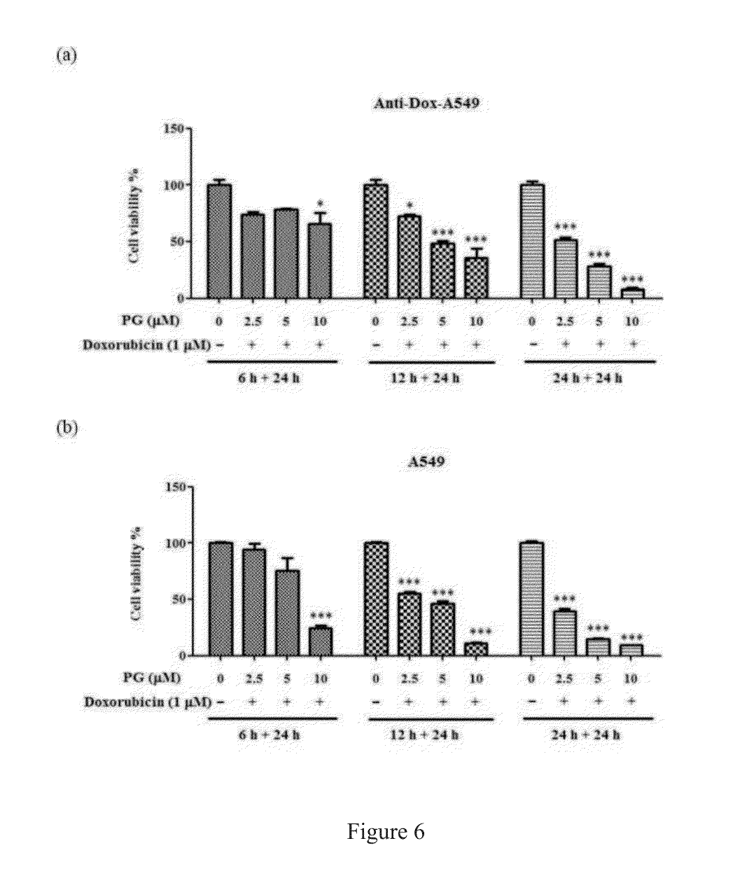

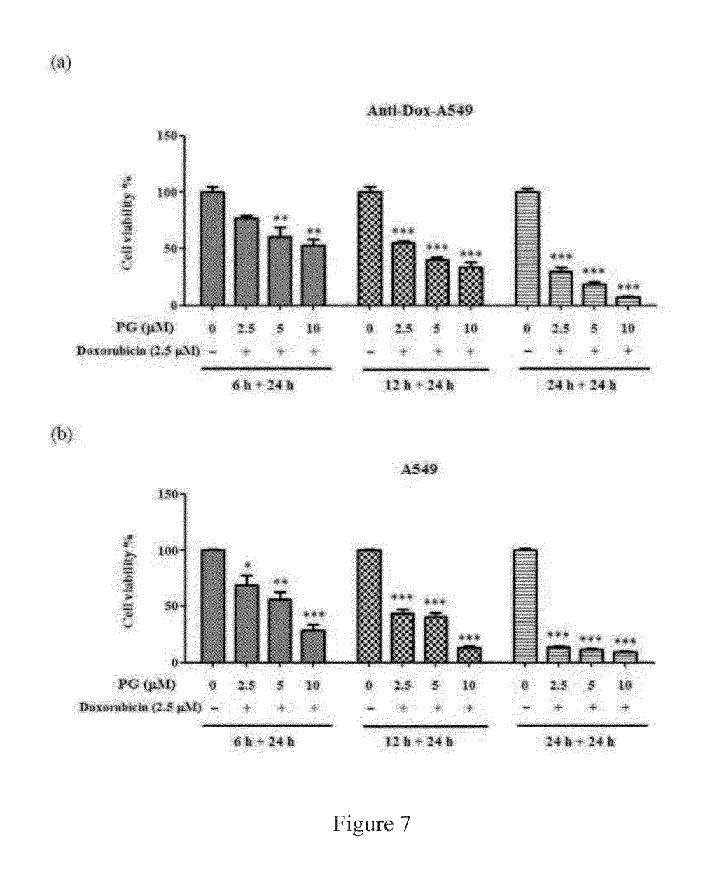

FIGS. 4 and 5 showed the effects of Dox treatment (1 μM and 2.5 μM, respectively) on cell viability after pre-treatment of HCD. The data indicated the potential that the pre-treatment of HCD (especially, at dosage of 10 or 25 μM) can enhance the effects of Dox on viability of both A549 cells and Anti-Dox-A549 cells even at low dosage (1 μM), which means the pre-treatment can rescue the sensitivity of Anti-Dox-A549 cells to Dox. Similar pattern showed in the experiments of PG pre-treatment (FIGS. 6 and 7). Accordingly, the pre-treatment strategies may be a practical therapy in clinical use.

In light of the foregoing, HCD showed reliable effects on the viability of lung cancer cells, including non-drug resistant and drug-resistant strains. Furthermore, our data indicated a pre-treatment strategy with a pre-treatment of HCD or PG before Dox treatment provided improved effects on lung cancer cells viability.

### EXAMPLE 3

### Cell Cycle Analysis

The results of Example 2 in cell viability hinted the effects of HCD, PG, and Dox on arresting cell cycle. In this example, the phase of cell cycle of A549 cells and Anti-Dox-A549 cells after the treatment listed in Table 1 was determined by flow cytometer. Briefly, 7×10⁴ cells per well were to inoculated in 12-wells plate and incubated overnight at 37° C., 5% CO₂. Then, cells were treated according to the treatment listed in Table 1 by indicated time period. After treatment, cells were harvested by trypsin and fixed with 70% ethanol at −20° C. for at least 3 hr. The cells were washed in cold PBS twice and then incubated with 1 ml (v/v) staining solution (20 μg/m1 propidium iodide (PI), 0.1% Triton X-100, 0.2 mg/ml Rnase) at 37° C. for 30 minutes. Lastly, cells were analyzed by flow cytometer (Cytomics™ FC500, Backman, Fullerton, Calif., USA). Data from 10,000 cells were collected for each experimental group.

The following Tables 2 to 5 showed that both of the treatment of HCD and PG increased cell cycle arrest at G2/M phase comparing with the control group. There was no significant difference between A549 cells and Anti-Dox-A549 cells and the tendency was more obvious at long treating time period (the 48 hr treatment in Table 2 and Table 3; 24 hr treatment in Table 4 and Table 5).

TABLE 2
Cell cycle distribution of Anti-Dox-A549 cells treated with various dosages of HCD.
Anti-Dox-A549

| Treatment | Sub G1 (%) | G0/G1 (%) | S (%) | G2/M (%) |
|---|---|---|---|---|
| 24 hr | | | | |
| Control | 0.83 ± 0.1 | 72.6 ± 2.5 | 10.5 ± 0.3 | 13.8 ± 1.9 |
| HCD2.5 | 0.63 ± 0.05 | 68.2 ± 2.8 | 12.1 ± 0.8 | 16.5 ± 1.8 |
| HCD10 | 0.73 ± 0.05 | 83.6 ± 1.4** | 7.0 ± 0.6 | 6.6 ± 0.55** |
| HCD25 | 1.3 ± 0.1** | 70.5 ± 2.7 | 7.9 ± 0.8** | 19.3 ± 1.7* |
| 36 hr | | | | |
| Control | 1.2 ± 0.1 | 82.5 ± 0.5 | 6.3 ± 0.2 | 8.7 ± 0.4 |
| HCD2.5 | 0.93 ± 0.2 | 80.3 ± 0.6 | 7.4 ± 0.2 | 9.6 ± 0.3 |
| HCD10 | 1 ± 0.2 | 75.8 ± 0.3** | 9.2 ± 0.4*** | 11.8 ± 1.0* |
| HCD25 | 3.43 ± 0.2*** | 73.9 ± 2.7*** | 5.4 ± 0.9 | 17.7 ± 1.9*** |
| 48 hr | | | | |
| Control | 1.8 ± 0.43 | 86.4 ± 0.6 | 5.3 ± 0.5 | 5.6 ± 0.5 |
| HCD2.5 | 1.56 ± 0.2 | 82.1 ± 0.7 | 6.1 ± 0.3 | 8.9 ± 0.7 |
| HCD10 | 1.76 ± 0.1 | 82.2 ± 0.7 | 6.4 ± 0.3 | 8.3 ± 0.2 |
| HCD25 | 27.2 ± 0.5*** | 3.73 ± 1.6*** | 11.2 ± 1.3*** | 21.8 ± 2.4*** |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.

TABLE 3
Cell cycle distribution of A549 cells treated with various dosages of HCD.
A549

| Treatment | Sub G1 (%) | G0/G1 (%) | S (%) | G2/M (%) |
|---|---|---|---|---|
| 24 hr | | | | |
| Control | 1 ± 0 | 68 ± 3.9 | 11.0 ± 0.5 | 15.2 ± 0.5 |
| HCD2.5 | 1.0 ± 0.2 | 77.3 ± 0.9 | 9.6 ± 0.5 | 9.7 ± 0.9 |
| HCD10 | 1 ± 0.2 | 85.6 ± 6.9** | 3.9 ± 2.8** | 6.1 ± 0.5*** |
| HCD25 | 1.5 ± 0.2 | 62.9 ± 3.3 | 11.1 ± 1.3 | 22.8 ± 3.0** |
| 36 hr | | | | |
| Control | 1.4 ± 0.2 | 79.7 ± 1.5 | 7.3 ± 0.6 | 9.4 ± 0.4 |
| HCD2.5 | 1.1 ± 0.1 | 74.4 ± 2.6* | 9.5 ± 0.4** | 11.8 ± 0.9 |
| HCD10 | 1.4 ± 0.2 | 75.4 ± 1.8 | 10.7 ± 0.4*** | 10.0 ± 1.2 |
| HCD25 | 2.7 ± 0.1*** | 62.3 ± 1.3*** | 9.2 ± 0.7* | 22.8 ± 1.5*** |
| 48 hr | | | | |
| Control | 2.1 ± 0.2 | 77.9 ± 6.6 | 6.6 ± 0.8 | 8.1 ± 0.25 |
| HCD2.5 | 1.8 ± 0.2 | 80.4 ± 0.2 | 6.9 ± 0.1 | 8.9 ± 0.1 |
| HCD10 | 1.8 ± 0.2 | 75.6 ± 1.8 | 8.7 ± 0.2 | 11.2 ± 0.2*** |
| HCD25 | 1.9 ± 0.5*** | 61.7 ± 1.2** | 7.9 ± 1.3 | 22.7 ± 0.5*** |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.

TABLE 4
Cell cycle distribution of Anti-Dox-A549 cells treated with various dosages of PG.
Anti-Dox-A549

| Treatment | Sub G1 (%) | G0/G1 (%) | S (%) | G2/M (%) |
|---|---|---|---|---|
| 6 hr | | | | |
| Control | 1.1 ± 0 | 66.1 ± 0.6 | 14.9 ± 0.2 | 17.2 ± 0.5 |
| PG2.5 | 2.2 ± 0.1* | 58.9 ± 7.1 | 18.5 ± 0.5 | 23.5 ± 1.9 |
| PG5 | 1.9 ± 0.3 | 56 ± 1.3 | 18.7 ± 0.8 | 23.6 ± 2.3 |
| PG10 | 2 ± 0.6 | 54.6 ± 3.0* | 22.9 ± 3.4** | 19.8 ± 6.6 |
| 12 hr | | | | |
| Control | 1.6 ± 1.2 | 72.3 ± 1.5 | 17.9 ± 3.1 | 8.0 ± 4.6 |
| PG2.5 | 1.6 ± 1.2 | 65.3 ± 2.9 | 13.8 ± 4.7 | 17.2 ± 6.5 |
| PG5 | 2.9 ± 1.0 | 62.7 ± 4.7* | 16.8 ± 5.5 | 19.2 ± 6.9 |
| PG10 | 1.8 ± 0.2 | 53.2 ± 2.5*** | 21.4 ± 3.7 | 22.8 ± 6.4 |
| 24 hr | | | | |
| Control | 0.9 ± 0.3 | 69.3 ± 4.3 | 15.0 ± 2.3 | 11.9 ± 3.0 |
| PG2.5 | 0.9 ± 0.3 | 81.8 ± 5.7 | 3.1 ± 1.3** | 8.8 ± 1.4 |
| PG5 | 4.3 ± 5.3 | 76.2 ± 6.0 | 3.7 ± 2.0** | 13.8 ± 1.7 |
| PG10 | 6.9 ± 4.3 | 44.3 ± 7.7** | 11.7 ± 3.7 | 27.9 ± 1.9*** |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.

TABLE 5
Cell cycle distribution of A549 cells treated with various dosages of PG.
A549

| Treatment | Sub G1 (%) | G0/G1 (%) | S (%) | G2/M (%) |
|---|---|---|---|---|
| 6 hr | | | | |
| Control | 0.26 ± 0.05 | 62 ± 2.4 | 13.6 ± 0.6 | 14.3 ± 11.7 |
| PG2.5 | 0.3 ± 0.1 | 46.3 ± 3.4*** | 17.3 ± 1.2** | 32.3 ± 3.1* |
| PG5 | 0.26 ± 0.05 | 42.9 ± 2.1*** | 18.2 ± 0.8*** | 34.8 ± 0.9* |
| PG10 | 0.33 ± 0.05 | 43.8 ± 3.1*** | 18.8 ± 0.5*** | 27 ± 2.9 |
| 12 hr | | | | |
| Control | 0.2 ± 0.1 | 65 ± 2 | 15.0 ± 0.3 | 14.2 ± 6.8 |
| PG2.5 | 0.4 ± 0.2 | 62.7 ± 2.7 | 6.6 ± 2.1** | 27.6 ± 0.7* |
| PG5 | 0.36 ± 0.2 | 53.3 ± 1.2*** | 10.0 ± 2.0* | 33.1 ± 0.2** |
| PG10 | 0.46 ± 0.1 | 41.6 ± 1.3*** | 16.6 ± 1.4 | 29.1 ± 3.0** |
| 24 hr | | | | |
| Control | 0.43 ± 0.2 | 64 ± 0.3 | 15.6 ± 0.2 | 19 ± 0.6 |
| PG2.5 | 0.26 ± 0.05 | 85 ± 0.6 | 1.9 ± 0.15*** | 11.6 ± 0.7 |
| PG5 | 0.23 ± 0.05 | 76.2 ± 8.1* | 3.0 ± 0.75*** | 22.2 ± 1.1 |
| PG10 | 0.9 ± 0.7 | 43.8 ± 2.4** | 9.1 ± 1.7*** | 26.5 ± 10.0 |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.

In comparison with the effect of HCD and PG, Anti-Dox-A549 cells treated with Dox (5, 10, or 20 μM) showed increased at Sub G1 phase and showed cell arrest at S phase (Table 6); in addition, A549 cells treated with Dox (5, 10, or 20 μM) also showed increased at Sub G1 phase and showed cell arrest at sub G1 phase (Table 7).

TABLE 6
Cell cycle distribution of Anti-Dox-A549
cells treated with various dosages of Dox.
Anti-Dox-A549

| Treatment | | | | |
|---|---|---|---|---|
| 24 hr | Sub G1 (%) | G0/G1 (%) | S (%) | G2/M (%) |
| Control | 1.3 ± 0.3 | 63.9 ± 3.8 | 14.1 ± 1.1 | 18.2 ± 4.3 |
| Dox5 | 6.4 ± 0.6*** | 54.7 ± 1.3* | 24.1 ± 0.5*** | 9.9 ± 0.7* |
| Dox10 | 4.6 ± 0.3*** | 62.8 ± 0.6 | 19.9 ± 0.1*** | 9.4 ± 0.9* |
| Dox20 | 6.2 ± 0.6*** | 58.1 ± 3.4 | 18.7 ± 0.4*** | 12.4 ± 1.5 |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.

TABLE 7
Cell cycle distribution of Anti-Dox-A549
cells treated with various dosages of Dox.
Anti-Dox-A549

| Treatment | | | | |
|---|---|---|---|---|
| 24 hr | Sub G1 (%) | G0/G1 (%) | S (%) | G2/M (%) |
| Control | 1.6 ± 0.2 | 61 ± 0.6 | 14.4 ± 0.6 | 20.1 ± 0.2 |
| Dox5 | 5.8 ± 0.5* | 50.3 ± 0.9*** | 23.5 ± 0.7*** | 15.2 ± 1.6** |
| Dox10 | 7.4 ± 2.3** | 50.2 ± 1.4*** | 20.3 ± 0.9*** | 17 ± 0.3* |
| Dox20 | 9.0 ± 0.8*** | 37.3 ± 2.1*** | 23.5 ± 0.7*** | 21.5 ± 1.6 |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.

In terms of the pre-treatment strategy, pre-treatment of HCD or PG and subsequent of Dox increased sub G1 phase and arrested at G2/M phase no matter in A549 cells or in Anti-Dox-A549 cells. The tendency was more obvious in higher dosage (HxD2.5, HyD2.5, HzD2 5, PxD2.5, PyD2.5, PzD2.5) (Tables 8 to 11). The results were consistent with the data obtained in Example 2, showing that the pre-treatment strategy is applicable for enhancing the effects of known drug, Dox.

TABLE 8
Cell cycle distribution of Anti-Dox-A549 cells pre-treated
with various dosages of HCD and then treated with Dox.
Anti-Dox-A549

| Treatment | Sub G1 (%) | G0/G1 (%) | S (%) | G2/M (%) |
|---|---|---|---|---|
| 6 hr + 24 hr | | | | |
| Control | 1 ± 0.3 | 69.3 ± 4.1 | 9.7 ± 0.9 | 16.2 ± 2.5 |
| HxD1 | 5.5 ± 2.1* | 44.1 ± 7.2** | 4.3 ± 0.2* | 43.7 ± 5.8*** |
| HyD1 | 5.1 ± 1.6* | 49.2 ± 5.8** | 6.6 ± 1.5 | 36.9 ± 4.9** |
| HzD1 | 3.7 ± 0.9 | 45.8 ± 0.2** | 17.2 ± 1.6** | 29.8 ± 2.4* |
| 12 hr + 24 hr | | | | |
| Control | 0.96 ± 0.32 | 6.94 ± 2.75 | 9.8 ± 1.34 | 15.8 ± 1.7 |
| HxD1 | 4.16 ± 1.32 | 61.1 ± 2.45 | 4.26 ± 1.65* | 29 ± 1.05 |
| HyD1 | 4.06 ± 1.89 | 67.5 ± 3.65 | 4.3 ± 1.91* | 24 ± 2.57 |
| HzD1 | 7.26 ± 5.7 | 54.1 ± 7.55* | 8.5 ± 1.4 | 21.1 ± 11.8 |
| 24 hr + 24 hr | | | | |
| Control | 1.3 ± 0.3 | 72.5 ± 8.82 | 7.66 ± 3.9 | 12 ± 0.78 |
| HxD1 | 4.66 ± 0.9 | 56.3 ± 3.5 | 2.53 ± 1.1 | 34.7 ± 0.9*** |
| HyD1 | 4.43 ± 0.15 | 62.3 ± 0.3 | 4.66 ± 2.2 | 27.4 ± 2.6** |
| HzD1 | 13.5 ± 18.3 | 66.7 ± 23.05 | 4.3 ± 2.49 | 17.6 ± 7.09 |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

TABLE 9
Cell cycle distribution of A549 cells pre-treated with
various dosages of HCD and then treated with Dox.
A549

| Treatment | Sub G1 (%) | G0/G1 (%) | S (%) | G2/M (%) |
|---|---|---|---|---|
| 6 hr + 24 hr | | | | |
| Control | 1.5 ± 0.3 | 70.6 ± 3.9 | 10.5 ± 1.4 | 14.3 ± 1.7 |
| HxD1 | 5.0 ± 0.8* | 46.3 ± 6.3** | 11.2 ± 1.1 | 30.2 ± 5.4** |
| HyD1 | 5.2 ± 1.0* | 50 ± 6.7** | 11.6 ± 0.9 | 25 ± 1.8* |
| HzD1 | 6.6 ± 1.9* | 45.3 ± 3.9** | 20.6 ± 4.3** | 23.0 ± 1.7* |
| 12 hr + 24 hr | | | | |
| Control | 4.1 ± 5.4 | 71.4 ± 7.0 | 8.8 ± 1.5 | 13.1 ± 1.6 |
| HxD1 | 11.5 ± 11.5 | 53.0 ± 5.4 | 7.8 ± 1.7 | 25.7 ± 6.2* |
| HyD1 | 10.6 ± 12.1 | 61.1 ± 10.7 | 6.3 ± 1.2 | 19.1 ± 3.9 |
| HzD1 | 21.1 ± 15.5 | 44.3 ± 14.2* | 11.5 ± 3.0 | 19.4 ± 5.6 |
| 24 hr + 24 hr | | | | |
| Control | 1.5 ± 0.4 | 75.7 ± 3.9 | 6.5 ± 0.6 | 10.2 ± 2.1 |
| HxD1 | 9.1 ± 4.2 | 59.1 ± 2.3* | 5.8 ± 1.0 | 24.2 ± 4.3** |
| HyD1 | 4.9 ± 0.9 | 70.3 ± 2.5 | 5.6 ± 1.6 | 15.9 ± 1.9 |
| HzD1 | 21.1 ± 3.9*** | 50.1 ± 8.1*** | 5.6 ± 1.6 | 15.9 ± 1.9 |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

TABLE 10
Cell cycle distribution of Anti-Dox-A549 cells pre-treated
with various dosages of PG and then treated with Dox.
Anti-Dox-A549

| Treatment | Sub G1 (%) | G0/G1 (%) | S (%) | G2/M (%) |
|---|---|---|---|---|
| 6 hr + 24 hr | | | | |
| Control | 0.8 ± 0.1 | 55.5 ± 14.7 | 9.9 ± 1.1 | 15.5 ± 1.4 |
| PxD1 | 6.5 ± 1.9 | 45.3 ± 2.9 | 4.7 ± 0.5 | 41.0 ± 4.9** |
| PyD1 | 6.4 ± 0.9 | 39.2 ± 4.8 | 7.9 ± 1.4 | 43.4 ± 4.4*** |
| PzD1 | 15.6 ± 4.9*** | 38.0 ± 7.7 | 17.7 ± 6.6 | 25.1 ± 7.5 |
| 12 hr + 24 hr | | | | |
| Control | 0.83 ± 0.2 | 72.6 ± 2.3 | 9.3 ± 1.4 | 14.1 ± 1.1 |
| PxD1 | 9.8 ± 3.8* | 63.7 ± 4.5 | 2.9 ± 0.2** | 21.5 ± 1.8** |
| PyD1 | 8.7 ± 1.5 | 46.1 ± 1.6*** | 5.6 ± 1.8 | 36.7 ± 2.1*** |
| PzD1 | 18.4 ± 5.4*** | 36.7 ± 6.2*** | 11.7 ± 1.6 | 30.3 ± 2.2*** |
| 24 hr + 24 hr | | | | |
| Control | 6.5 ± 9.8 | 72.1 ± 16.6 | 12.1 ± 12.7 | 10.6 ± 1.0 |
| PxD1 | 27.1 ± 8.0 | 59.5 ± 11.2 | 1.6 ± 0.3 | 5.6 ± 2.1 |
| PyD1 | 26.4 ± 5.6 | 55.1 ± 10.8 | 4.1 ± 1.4 | 12.9 ± 3.5 |
| PzD1 | 38.7 ± 8.4** | 32.9 ± 10.6* | 8 ± 1.0 | 18.8 ± 0.8** |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

TABLE 11
Cell cycle distribution of A549 cells pre-treated with
various dosages of PG and then treated with Dox.
A549

| Treatment | Sub G1 (%) | G0/G1 (%) | S (%) | G2/M (%) |
|---|---|---|---|---|
| 6 hr + 24 hr | | | | |
| Control | 0.73 ± 0.1 | 63.6 ± 4.17 | 11.6 ± 0.7 | 16.9 ± 0.9 |
| PxD1 | 7.6 ± 2.4 | 41.1 ± 3.8*** | 9.9 ± 4.0 | 39.0 ± 5.1** |
| PyD1 | 6.6 ± 2.8 | 39.1 ± 6.4*** | 10.6 ± 2.4 | 41.4 ± 6.2*** |
| PzD1 | 12.8 ± 3.8** | 33.4 ± 0.1*** | 16.3 ± 1.2 | 34.5 ± 3.2** |
| 12 hr + 24 hr | | | | |
| Control | 1.5 ± 0.6 | 69.6 ± 5.8 | 10.4 ± 3.1 | 14.1 ± 1.7 |
| PxD1 | 12.6 ± 3.5** | 64.5 ± 4.0 | 4.2 ± 0.6 | 17.0 ± 4.6 |
| PyD1 | 12.5 ± 2.7* | 50.5 ± 6.0** | 7.6 ± 2.7 | 27.3 ± 7.0 |
| PzD1 | 20.2 ± 4.2*** | 34.2 ± 3.7*** | 12.7 ± 3.5 | 29.8 ± 5.7* |
| 24 hr + 24 hr | | | | |
| Control | 1.54 ± 0.8 | 70.9 ± 13.3 | 13.2 ± 12.2 | 9.06 ± 1.1 |
| PxD1 | 32.0 ± 6.6*** | 60.0 ± 6.4 | 2.16 ± 0.2 | 4.3 ± 1.0** |
| PyD1 | 35.2 ± 9.4*** | 51.9 ± 10.4 | 3.06 ± 0.6 | 8.1 ± 0.1 |
| PzD1 | 45.2 ± 0.9*** | 27.3 ± 2.4** | 19.2 ± 1.9 | 19.2 ± 1.9*** |

Note:
1. Control group: untreated cells.
2. All the experiments were done in three independent trails and the data present as mean ± SD in triplicate determinations. Compared at
*p < 0.05,
**p < 0.01,
***p < 0.01 levels with untreated control.
3. 6 hr + 24 hr: 6 hr of pre-treatment and 24 hr of subsequent treatment.
4. 12 hr + 24 hr: 12 hr of pre-treatment and 24 hr of subsequent treatment.
5. 24 hr + 24 hr: 24 hr of pre-treatment and 24 hr of subsequent treatment.

### EXAMPLE 4

### Mouse Model Establishment

Previously in the field, mouse models (nude or SCID, severe combined immunodeficiency) bearing human lung cancer (xenograft) have been reported and accepted in cancer researches. However, those models are not ideal because the mice bear human lung cancer in back or thigh (non-in situ) instead of in lung (in situ); therefore, the pathophysiological condition is a lot different from a human body. In order to test the HCD's efficacy on lung cancer treatment in a more realistic condition, this example was to establish a normal mouse model bearing human lung cancer in lung.

The animal experiments were approved by the National Dong-Hwa University Animal Ethics Committee and the experimental protocols were used according to the “Guide for the Care and Use of Laboratory Animals” of National Dong-Hwa University. 1×10⁷ of A549 cells or Anti-Dox-A549 cells were suspended in PBS to prepare cell suspensions. On day 0, C57/BL6 mice (6-8 weeks old, n=6) were weighted and anesthetic using pentobarbital (5 mg/ml). Then, the mice were injected with the cell suspension (1×10⁷ cells per mouse) into the lung by IT. The injection was repeated every 7 days for 21 days. Another group of mice were injected with 10% EtOH as comparative group according to the same procedure set forth above.

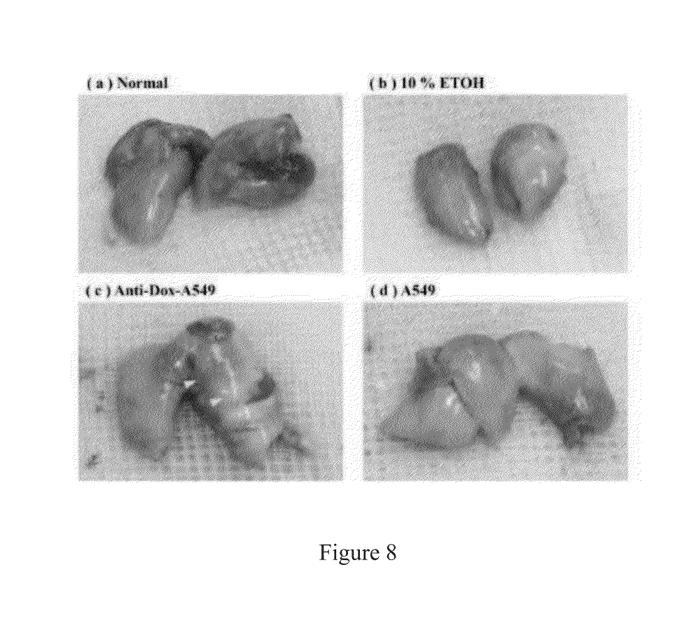

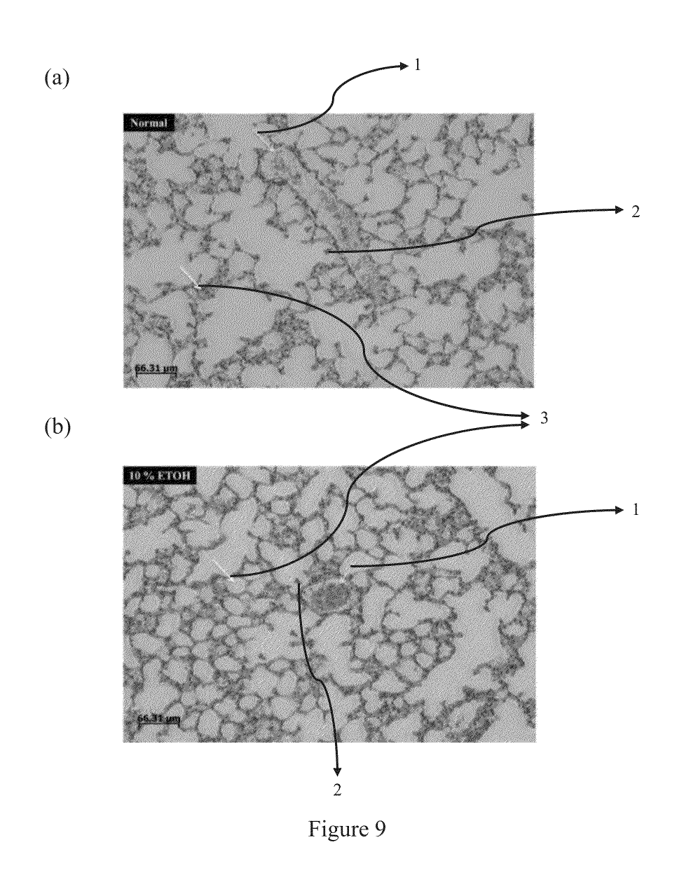

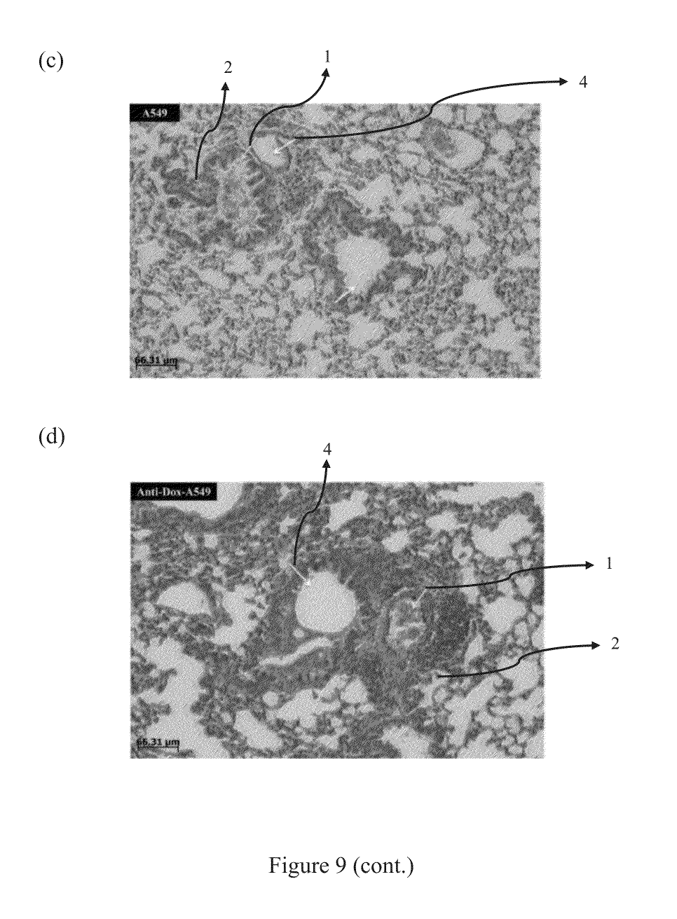

Afterward, the mice were sacrificed at day 28, and the lungs were obtained to investigate the morphology thereof. There were more pale patches distributed in the lung of mouse injected with A549 cells (FIG. 8d) or Anti-Dox-A549 cells (FIG. 8c) than normal mouse (FIG. 8a) and mouse injected with 10% EtOH (FIG. 8b). In addition, the lungs were examined by H&E and IHC staining for checking human lung adenocarcinoma markers. The H&E staining confirmed that, in mouse injected with A549 cells or Anti-Dox-A549 cells, there were more cells invasion occurred around blood vessels 1 and more cell accumulation 2 around the trachea 4 (FIG. 9), showing carcinogenesis happened. Accordingly, the method recited in this example successfully established a mouse model bearing human lung cancer.

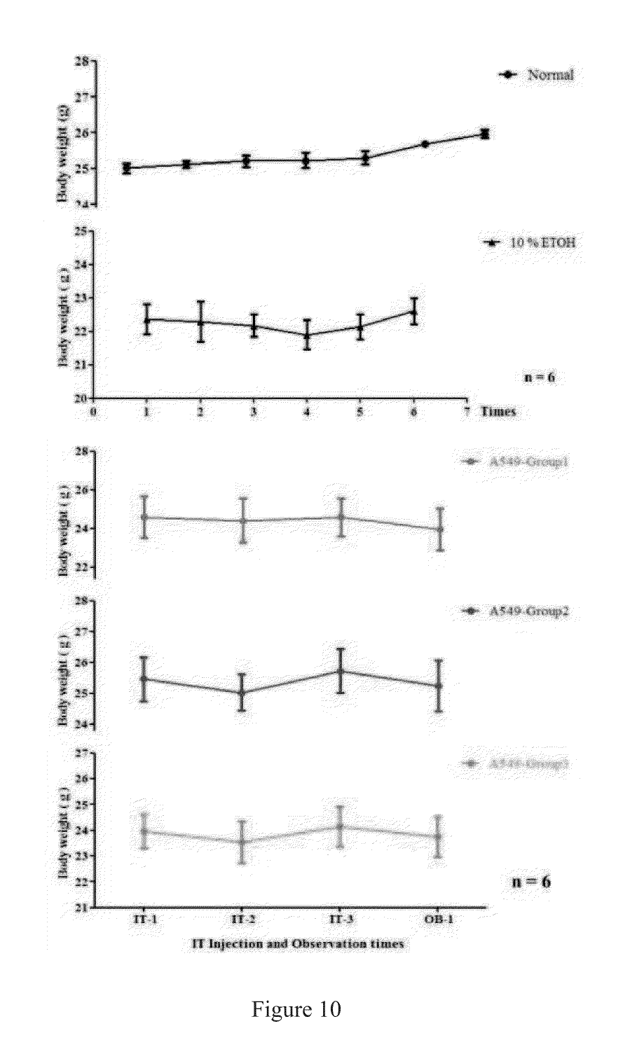

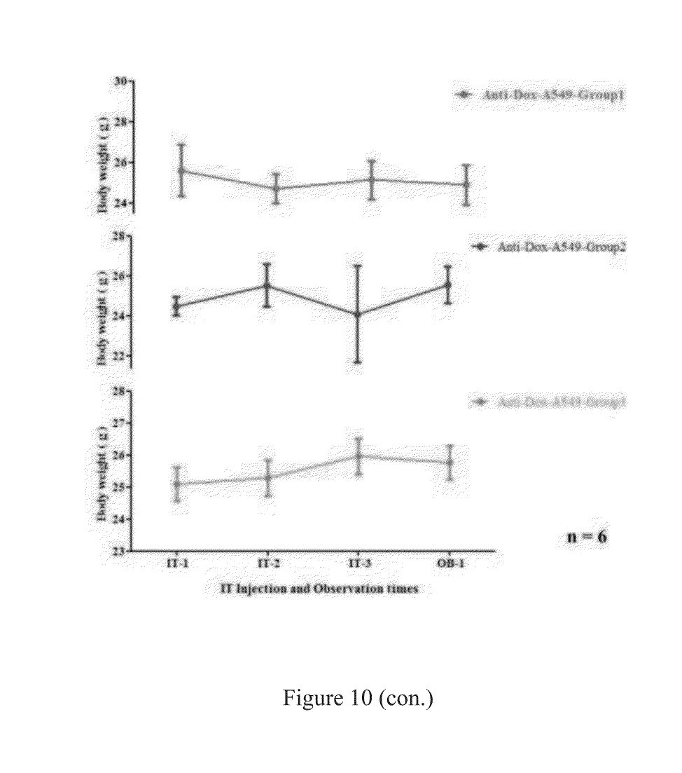

Furthermore, the body weight was also recorded to see if the injection caused any growth defect. FIG. 10 showed the body weight is changing curves of normal mice (uninjected), mice injected with 10% EtOH, and mice injected with A549 cells suspension or Anti-Dox-A549 cells suspension. The results showed that the body weight made no change during the injection, indicating the growth condition of the experimental mice was stable and the established mouse model shall be reliable for subsequent studies.

### EXAMPLE 5

### Examination to the Efficacy of HCD in Lung Cancer Treatment by Mouse Model.

After the establishment of human lung cancer in the experimental mice, the mice were maintained for one more week and then treated with HCD (0.0318 or 1.272 mg/kg body weight, in 10% ETOH with PBS), PG (0.1615 or 0.646 mg/kg body weight, in 10% ETOH with PBS) or 10% EtOH by IT injection (intrathecal injection) to the lung. The body weight was monitored every five days for thirty days. After thirty days, the mice were sacrificed and the lungs were checked for marker of human lung adenocarcinoma by H&E and IHC staining. The livers, kidneys and spleens of the mice were also examined to confirm the cytotoxicity of HCD and PG and to see if there was any metastasis happened. The results showed that there was no metastasis happened in livers, kidneys and spleens of the mice (data not shown). In addition, the livers, kidneys and spleens were not injured from the staining showing HCD did not cause serious cytotoxicity (data not shown).

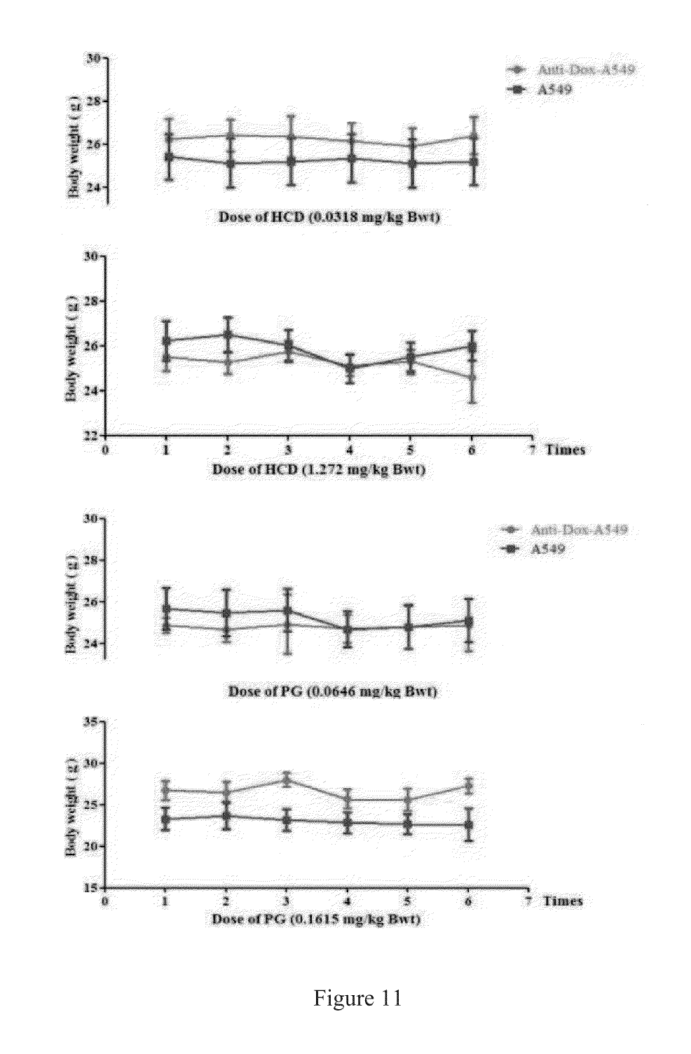

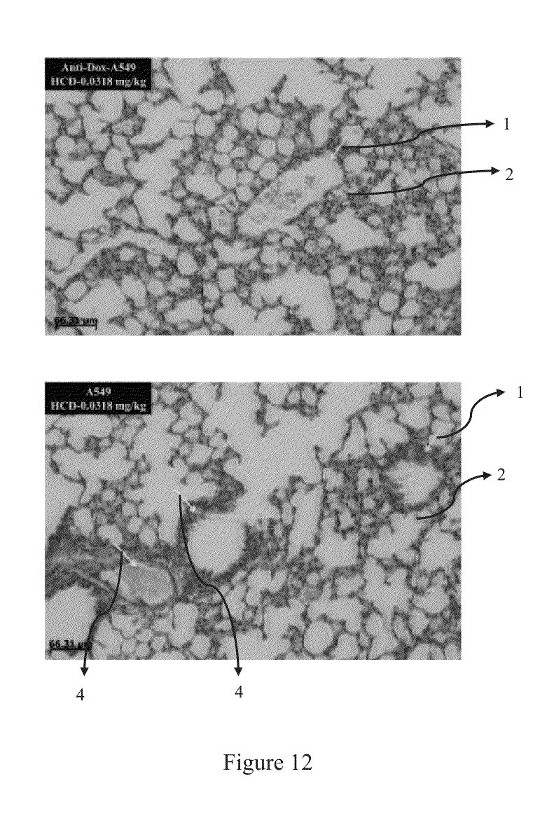

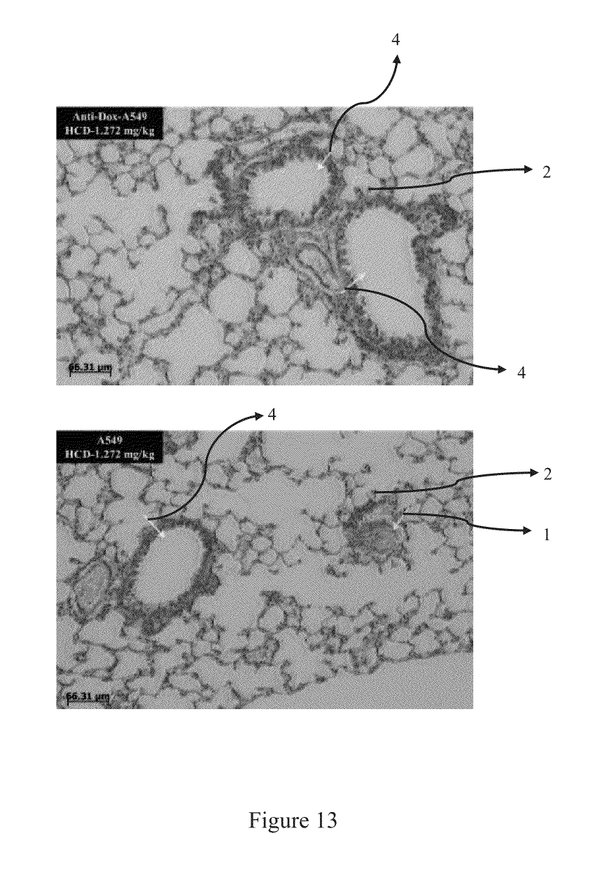

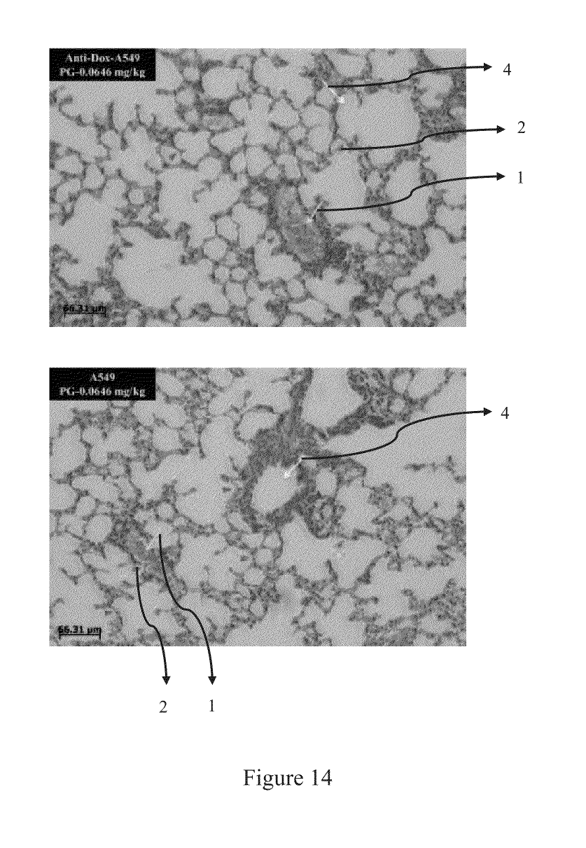

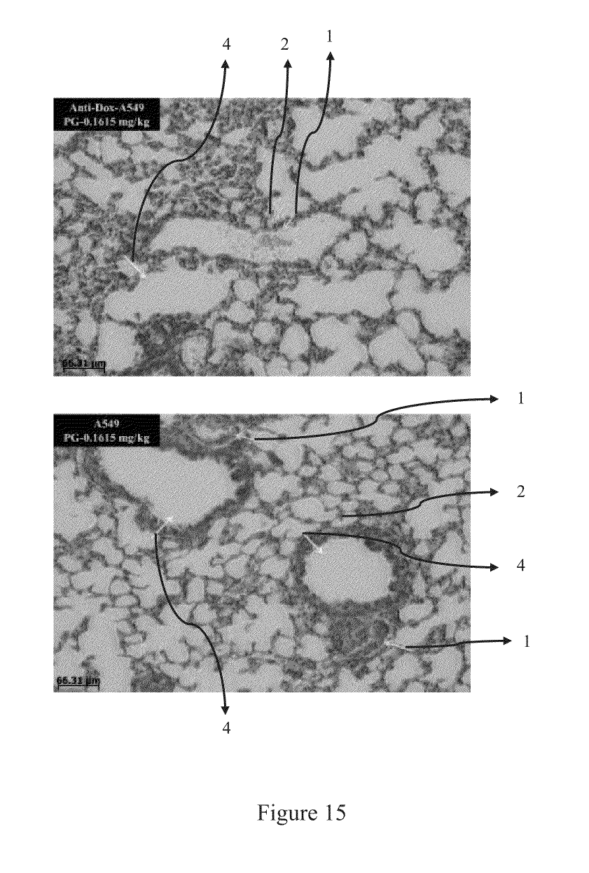

According to FIG. 11, in both of the HCD and PG treatment, the to body weight of the experimental mice did not show significantly change during the experimental period. With regard to the H&E and IHC staining, the lung of the mouse injected showed less cell invention around blood vessels 1 and less cell accumulation 2 around the trachea 4 (FIG. 12 and FIG. 13). Similar results showed in the PG treatment. There were less cell invention around blood vessels 1 and less cell accumulation 2 around the trachea 4 (FIG. 14 and FIG. 15). The data confirmed the efficacy of HCD and PG in lung cancer treatment and was consistent with the data obtained in the above in vitro studies.

### REFERENCE

Liu, F., D. Liu, Y. Yang and S. Zhao (2013). “Effect of autophagy inhibition on chemotherapy-induced apoptosis in A549 lung cancer cells.” Oncol Lett 5(4):

1261-1265

Lu, C., C. Shao, E. Cobos, K. P. Singh and W. Gao (2012). “Chemotherapeutic sensitization of leptomycin B resistant lung cancer cells by pretreatment with doxorubicin.” PLoS One 7(3): e32895.

Thiyagarajan, V., S. H. Lin, Y. C. Chia and C. F. Weng (2013). “A novel to inhibitor,16-hydroxy-cleroda-3,13-dien-16,15-olide, blocks the autophosphorylation site of focal adhesion kinase (Y397) by molecular docking.” Biochim Biophys Acta 1830(8): 4091-4101.

Zhou, W., Z. X. Jin and Y. J. Wan (2010). “Apoptosis of human lung adenocarcinoma A549 cells induced by prodigiosin analogue obtained from is an entomopathogenic bacterium Serratia marcescens.” Appl Microbiol Biotechnol 88(6): 1269-1275.

Li, D. D., J. F. Guo, J. J. Huang, L. L. Wang, R. Deng, J. N. Liu, G. K. Feng, D. J. Xiao, S. Z. Deng, X. S. Zhang and X. F. Zhu (2010). “Rhabdastrellic acid-A induced autophagy-associated cell death through blocking Akt pathway in human cancer cells.” PLoS One 5(8): e12176.

Ling, Y. H., M. Aracil, Y. Zou, Z. Yuan, B. Lu, J. Jimeno, A. M. Cuervo and R. Perez-Soler (2011). “PMO2734 (elisidepsin) induces caspase-independent cell death associated with features of autophagy, inhibition of the Akt/mTOR signaling pathway, and activation of death-associated protein kinase.” Clin Cancer Res 17(16): 5353-5366.

What is claimed is:

1. A pharmaceutical composition for lung cancer treatment, comprising:
an effective amount of 16-hydroxy-cleroda-3,13-dine-15,16-olide; and
a pharmaceutically acceptable carrier.

2. The composition of claim 1, wherein said effective amount is 0.01 to 1.28 mg/kg body weight.

3. The composition of claim 1, comprising 2.1 to 25 μM of said 16-hydroxy-cleroda-3,13-dine-15,16-olide.

4. The composition of claim 1, wherein said lung cancer is squamous cell carcinoma, large-cell lung cancer, small-cell lung cancer, non-small-cell lung cancer, or a combination thereof.

5. The composition of claim 1, wherein said lung cancer is resistant to Doxorubicin.

6. The composition of claim 1, wherein said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

7. The composition of claim 1, wherein an administration route of said composition is via oral administration, intravenous injection, intrathecal injection, or a combination thereof.

8. A method for treating a lung cancer, comprising:
administrating an object in need an effective amount of 16-hydroxy-cleroda-3,13-dine-15,16-olide.

9. The method of claim 8, wherein said effective amount is 0.01 to 1.28 mg/kg body weight.

10. The method of claim 8, wherein said administrating is via oral administration, intravenous injection, intrathecal injection, or a combination thereof.

11. The method of claim 8, wherein said 16-hydroxy-cleroda-3,13-dine-15,16-olide is administrated with a pharmaceutically acceptable carrier

12. The method of claim 8, wherein said pharmaceutically acceptable carrier is water, phosphate buffered saline, alcohol, glycerol, chitosan, alginate, chondroitin, Vitamin E, mineral oil, dimethyl sulfoxide (DMSO), or a combination thereof.

13. The method of claim 8, further comprising a step after administrating said 16-hydroxy-cleroda-3,13-dine-15,16-olide: administrating said object with Doxorubicin.

14. The method of claim 8, wherein said lung cancer is squamous cell carcinoma, large-cell lung cancer, small-cell lung cancer, non-small-cell lung cancer, or a combination thereof.

15. A method for establishing an animal model bearing human lung cancer:
providing an animal;
providing a cell suspension; wherein said cell is human lung cancer cell; and
administrating said cell suspension into said animal's lung.

16. The method of claim 15, wherein said animal is not immunodeficient.

17. The method of claim 15, wherein said human lung cancer cell is a commercially-available cell line.

18. The method of claim 15, wherein said human lung cancer cell is resistant to an anti-cancer drug.

19. The method of claim 18, wherein said anti-cancer drug is Doxorubicin.

20. The method of claim 15, wherein said administrating is via intrathecal injection.

21. The method of claim 15, wherein said administrating is conducted with a dosage of 1×10⁵ to 1×10⁷ said cells per animal.

22. The method of claim 15, wherein said animal is rabbit, pig or rodent.

23. The method of claim 15, wherein said administrating is repeated at least once.
